(19) United States
(12) Patent Application Publication
(10) Pub. No.: US 2025/0340903 A1
(43) Pub. Date: Nov. 6, 2025

(54) Modified Herpes Simplex Virus Type 1

(71) Applicant: EG 427, Paris (FR)

(72) Inventors: Grzegorz F. SAREK, Basel (CH); Tomasz BENEDYK, Tychy (PL); Yohann DICKX, Paris (FR)

(21) Appl. No.: 19/233,557

(22) Filed: Jun. 10, 2025

Related U.S. Application Data
(63) Continuation of application No. PCT/EP2023/085621, filed on Dec. 13, 2023.

(30) Foreign Application Priority Data
Dec. 14, 2022 (FR) 2213398

(51) Int. Cl. C12N 15/86

(52) U.S. Cl. CPC C12N 15/86; C12N 2710/16022; C12N 2710/16043; C12N 2710/16052; C12N 2710/16062

(57) ABSTRACT

This application describes a modified herpes simplex virus type 1 (HSV-1), capable of being efficiently produced in suspension cell culture, and a method of producing HSV-1 vectors in suspension cell culture.

### FIELD OF THE APPLICATION

This application relates to a modified herpes simplex viruses type 1 (HSV-1) vector, wherein the modification allows for the modified HSV-1 vector to be efficiently produced in suspension cell lines.

### BACKGROUND

Virus vectors have been employed as gene transfer vehicles for various preclinical and clinical gene therapy applications. Among them, herpes simplex viruses type 1 (HSV-1) vector is especially suitable for treating diseases affecting the central nervous system (CNS), such as Parkinson's disease or malignant gliomas, due to its advantageous properties including natural neurotropism, high transduction efficiency, large transgene capacity, and the ability of entering a latent state in neurons. In fact, Imlygic® (T-VEC, Talimogene laherparepvec), constituted by an engineered HSV-1, has become the first FDA-approved viral gene therapy product used in standard patient care. In addition to the replication-competent oncolytic HSV-1 vectors like T-VEC, replication-defective HSV-1 vectors have been explored as delivery vehicles for gene therapies targeting genetic skin diseases, disorders such as pain, neuropathy, and other neurodegenerative conditions. During the last decade, research on developing HSV-1 vectors has led to the recombinant HSV-1 vectors that are nontoxic and capable of long-term transgene expression in neurons.

However, although HSV-1 vector emerging as an effective and powerful therapeutic approach, the manufacture of HSV-1 vectors remains a challenging issue, limiting their speed to market. Currently, the production of HSV-1 vectors is relying on adherent cell lines (such as the Vero cell line), which require cells to be disassociated from their growth surface for routine maintenance, scale-up, and counting. Cell growth is restricted to surface area in adherent cell culture and hence the product yields are essentially limited. In contrast, in suspension cell culture, infected cells for producing viral vectors are not dependent on a surface for growth and thus scale-up becomes much easier. Therefore, given the manufacturing capacities, it will be advantageous to modify HSV-1 vectors to render them capable of being stably produced in suspension cell lines.

As HSV-1 vector becomes an increasingly attractive vehicle for gene therapy, there is a growing need for more efficient manufacturing process. A new generation of HSV-1 vectors that is capable of being produced in suspension cell lines will allow optimization of the manufacture, improve the vector yields, and meet the needs of various therapeutic, prophylactic, and research applications.

### SUMMARY OF THE INVENTION

This application provides a modified HSV-1 vector that is capable of being produced in suspension cell lines, such as CHO, HeLa, H-9, Jurkat, C6/36, High Five, S2, Sf21, Sf9, PC-1, and HEK293. The modified HSV-1 vector comprises a mutation, wherein the mutation is a single nucleotide substitution in the coding sequence of US2, or a single nucleotide substitution in the coding sequence of US8. Preferably, the single nucleotide substitution at position 827 in the coding sequence of US2 results in a G276V mutation, which 827 position is numbered relative to the position in the wild-type US2 gene (SEQ ID NO: 1). More preferably, the coding sequence of US2 after substitution comprises a nucleic acid sequence of SEQ ID NO: 2. Preferably, the single nucleotide substitution at position 348 in the coding sequence of US8 results in a W116STOP mutation, which 348 position is numbered relative to the position in the wild-type US8 gene (SEQ ID NO: 3). More preferably, the coding sequence of US8 after substitution comprises a nucleic acid sequence of SEQ ID NO: 4.

In embodiments, the single nucleotide substitution in US8 leading to an early stop codon can be at any other position within the region of the US8 gene coding for the extracellular domain of the US8 protein.

In embodiments, the single nucleotide substitution in US8 leading to an early stop codon can be at any other position within the region of the US8 gene coding for the transmembrane domain of the US8 protein that results in protein comprising a nonfunctional transmembrane domain.

This application also provides a modified HSV-1 vector comprising an inactivating deletion of US8 (gE) gene. The inactivating deletion can be a deletion within, or of, the entire coding sequence of the US8 (gE) gene or alternatively including the promoter or other regulatory sequences of such gene. In one aspect, the inactivating deletion can be a complete deletion of the coding sequence of the US8 (gE) gene, such that the virus genome does not contain nucleic acid sequences of the US8 (gE) gene. As used herein, an “inactivating deletion” of the US8 gene is any deletion that results in the absence of the US8 protein on the surface of the HSV-1 virus.

In embodiments, the inactivating deletion in US8 gene results in a truncated protein lacking the transmembrane domain or a functional transmembrane domain.

In embodiments, the inactivating deletion in US8 gene results in the modified HSV-1 vector lacking the US8 protein.

Without wishing to be bound by any particular theory, it is believed that a truncated US8 protein lacking the transmembrane domain or a functional transmembrane domain or the lack of the US8 protein results in a modified HSV-1 lacking the US8 protein in the viral envelope, which is required for efficient cell to cell spread.

In some embodiments, the inactivating deletion comprises the single nucleotide substitution in the coding sequence of US8 as described herein.

In some embodiments, before the introduction of the mutation, the parental HSV-1 vector is a wild-type HSV-1 vector, preferably the human HSV-1 strain F comprising the genome of GenBank Accession No. GU734771.1.

In some embodiments, before the introduction of the inactivating deletion of the US8 gene, the parental HSV-1 vector is a wild-type HSV-1 vector, preferably the human HSV-1 strain F comprising the genome of GenBank Accession No. GU734771.1.

In some embodiments, the term “modified HSV-1 vector” refers to a HSV-1 vector having the W116STOP mutation as described herein.

In some embodiments, the term “modified HSV-1 vector” refers to a HSV-1 vector having the inactivating deletion of the US8 gene.

In some embodiments, the modified HSV-1 vector is a recombinant HSV-1 vector, a replication competent HSV-1 vector, a defective helper-independent HSV-1 vector, a helper HSV-1 vector, or an HSV-1 amplicon vector.

In some embodiments, the modified HSV-1 vector is an HSV-1 amplicon vector, wherein a helper virus-dependent packaging system is used for the production of the HSV-1 amplicon vector, and wherein the helper virus-dependent system comprises a helper HSV-1 vector comprising the mutation. In some embodiments, the modified HSV-1 vector is an HSV-1 amplicon vector that comprise a mutant US2 protein, expressed by SEQ ID NO: 2, comprising a G276V mutation relative to the wild US2 protein, preferably present in the tegument of the HSV-1 amplicon vector. In some embodiments, the modified HSV-1 vector is an HSV-1 amplicon vector that comprise a mutant US8 protein (gE), expressed by SEQ ID NO: 4, that comprise a W116STOP mutation relative to the wild US8 protein (gE).

In some embodiments, the modified HSV-1 vector is an HSV-1 amplicon vector that comprise a modified US8 (gE) protein or lacks the US8 (gE) protein. In embodiments, the inactivating deletion of the US8 gene in the helper virus can result in a non-functional US8 protein in the HSV-1 amplicon vector. In embodiments, the inactivating deletion of the US8 gene in the helper virus can result in the lack of the US8 protein in the HSV-1 amplicon.

The modified HSV-1 vector may further comprise a genome comprising an exogenous expression cassette. The expression cassette may comprise at least one nucleic acid sequence encoding a gene product.

This application also provides a pharmaceutical composition comprising the modified HSV-1 vector as described herein, and a pharmaceutical excipient.

This application also provides a kit that comprises the modified HSV-1 vector as described herein and instructions.

This application also provides a method of manufacturing an HSV-1 vector in suspension cell lines, wherein the method comprises infecting a suspension cell line with the modified HSV-1 vector as described herein; and culturing the infected cells. This application also provides a method of manufacturing a modified HSV-1 amplicon vector in suspension cell lines, wherein the method comprises infecting a suspension cell line with a helper virus-dependent packaging system as described herein; and culturing the infected cells.

### BRIEF DESCRIPTION OF THE DRAWINGS

FIG. 1 depicts a wild-type HSV-1 (wt HSV-1) vector backbone and a mutant HSV-1 vector backbone. The wild-type HSV-1 vector backbone comprises a US2 coding sequence of SEQ ID NO: 1 and a US8 coding sequence of SEQ ID NO: 3. The mutant HSV-1 vector backbone comprises a G276V substitution in US2 coding sequence, resulting in a US2 coding sequence of SEQ ID NO: 2; and/or a W116STOP substitution in US8 (gE) coding sequence, resulting in a US8 coding sequence of SEQ ID NO: 4.

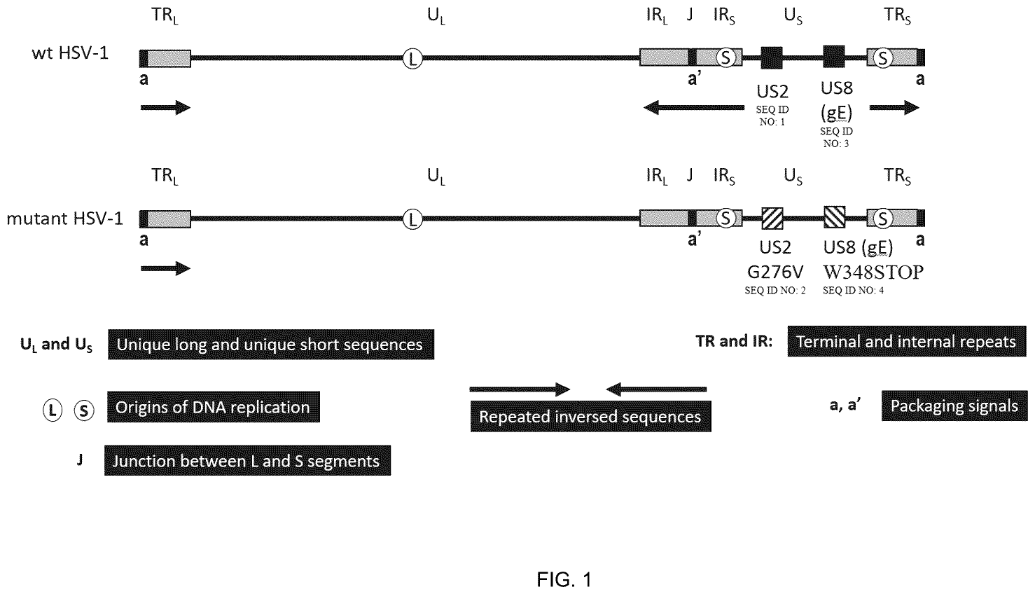

FIG. 2 depicts a modified non-replicative HSV-1 (modified nr HSV-1) vector backbone comprising a G276V substitution in US2 coding sequence, resulting in a US2 coding sequence of SEQ ID NO: 2, and/or a W116STOP substitution in US8 (gE) coding sequence, resulting in a US8 coding sequence of SEQ ID NO: 4. “A” represents genes that have been deleted, including Δjoint and ΔICP4 (second copy).

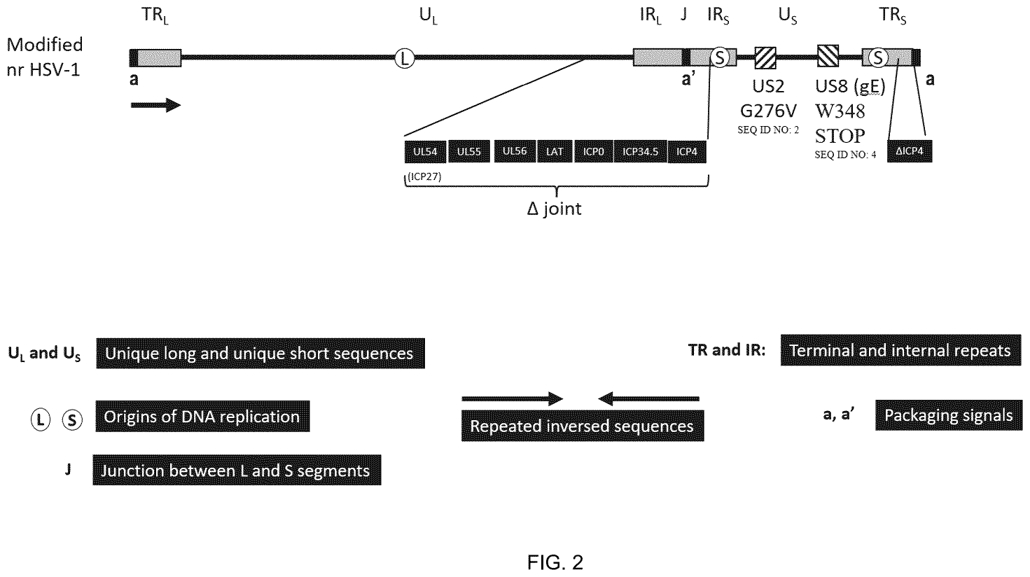

FIG. 3 depicts a parental helper HSV-1 vector backbone, that is used in a helper virus-dependent packaging system for the production of HSV-1 amplicon vector, wherein the two copies of ICP4 genes are deleted; and a modified helper HSV-1 vector backbone based on the parental helper HSV-1 vector backbone. The modified helper HSV-1 vector comprises a G276V substitution in US2 coding sequence, resulting in a US2 coding sequence of SEQ ID NO: 2; and/or a W116STOP substitution in US8 (gE) coding sequence, resulting in a US8 coding sequence of SEQ ID NO: 4.

FIG. 4A and FIG. 4B show that only the truncated US8 mutation W116STOP leads to improved manufacturing total yield of HSV-1 vectors in HEK293 in suspension.

FIG. 5A and FIG. 5B show that only the truncated US8 mutation W116STOP leads to improved manufacturing total yield of HSV-1 vectors in HEK293 in suspension.

FIG. 6A and FIG. 6B show that the truncated US8 mutation W116STOP does not impair manufacturing total yield of HSV-1 vectors in SH-SY5Y adherent cell line.

FIG. 7A and FIG. 7B show that the truncated US8 mutation W116STOP does not impair manufacturing total yield of HSV-1 vectors in Vero adherent cell line.

FIG. 8 shows that the HSV-1 tUS8 allows larger proportion of the produced particles available in the supernatant.

### DETAILED DESCRIPTION

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising”, which is used interchangeably with “including”, “containing”, or “characterized by”, is inclusive or open-ended language and does not exclude additional, unrecited elements or method steps.

The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” as used herein refers to any individual or patient to which the subject methods are performed. Generally, the subject is human, although as will be appreciated by those in the art, the subject may be an animal. Thus, other animals, including mammals such as rodents (including mice, rats, hamsters, and guinea pigs), cats, dogs, rabbits, farm animals including cows, horses, goats, sheep, pigs, etc., and primates (including monkeys, chimpanzees, orangutans and gorillas) are included within the definition of subject.

A “therapeutic effect,” as used herein, encompasses a therapeutic benefit and/or a prophylactic benefit as described herein.

As used herein, “treatment” or “treating” means to administer a composition to a subject or a system with an undesired condition. The condition can include a disease or disorder. “Prevention” or “preventing” means to administer a composition to a subject or a system at risk for the condition. The condition can include a predisposition to a disease or disorder. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “active fragment” refers to an amino acid fragment that is less than the entire amino acid sequence of the molecule and retains substantially the same biological activity or a corresponding biological activity, for example, an activity of more than 50%, such as 60%, 70%, 80%, 85%, 90%, 91%, 92%, 93%, 94%, 95%, 96%, 97%, 98%, 99% or 100%.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, a “regulatory gene” or “regulatory sequence” is a nucleic acid sequence that encodes products (e.g., transcription factors) that control the expression of other genes.

As used herein, a “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from eukaryotic mRNA, genomic DNA sequences from eukaryotic DNA, and synthetic nucleic acids. A transcription termination sequence will usually be located 3′ to the coding sequence.

The term “transgene” refers to a particular nucleic acid sequence encoding an RNA and/or a polypeptide or a portion of a polypeptide to be expressed in a cell into which the nucleic acid sequence is introduced. The term “transgene” includes (1) a nucleic acid sequence that is not naturally found in the cell (i.e., a heterologous nucleic acid sequence); (2) a nucleic acid sequence that is a mutant form of a nucleic acid sequence naturally found in the cell into which it has been introduced; (3) a nucleic acid sequence that serves to add additional copies of the same (i.e., homologous) or a similar nucleic acid sequence naturally occurring in the cell into which it has been introduced; or (4) a silent naturally occurring or homologous nucleic acid sequence whose expression is induced in the cell into which it has been introduced. By “mutant form” is meant a nucleic acid sequence that contains one or more nucleotides that are different from the wild-type or naturally occurring sequence, i.e., the mutant nucleic acid sequence contains one or more nucleotide substitutions, deletions, and/or insertions. In some cases, the transgene may also include a sequence encoding a leader peptide or signal sequence such that the transgene product will be secreted from the cell, or the transgene may include both a leader peptide or signal sequence plus a membrane anchor peptide, or even be a fusion protein between two naturally occurring proteins or part of them, such that the transgene will remain anchored to cell membranes, or a sequence that allows the protein to accumulate in a specific region of the cell, such as a nuclear localizing signal.

As used herein, the term “expression cassette” or “transcription cassette” refers to a distinct component of vector DNA consisting of a gene and regulatory sequence to be expressed by a transfected or transduced cell. In each successful transfection, the expression cassette directs the cell's machinery to make RNA and protein(s). Some expression cassettes are designed for modular cloning of protein-encoding sequences so that the same cassette can easily be altered to make different proteins. An expression cassette can be composed of one or more genes and the sequences controlling their expression. An expression cassette comprises at least three components: a promoter sequence, an open reading frame, and a 3′ untranslated region that, in eukaryotes, usually contains a polyadenylation site.

As used herein, a “promoter” is defined as a regulatory DNA sequence generally located upstream of a gene that mediates the initiation of transcription by directing RNA polymerase to bind to DNA and initiating RNA synthesis. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular compound or protein), it may be a spatially restricted promoter (i.e., transcriptional control element, enhancer, etc.; e.g., tissue specific promoter, cell type specific promoter, etc.), and it may be a temporally restricted promoter (i.e., the promoter is in the “ON” state or “OFF” state during specific stages of embryonic development or during specific stages of a biological process). For purposes of the present invention, a promoter sequence includes at least the minimum number of bases or elements necessary to initiate transcription of a gene of interest at levels detectable above background. Within the promoter sequence is a transcription initiation site, as well as RNA polymerase binding domains.

Eukaryotic promoters will often, but not always, contain “TATA” boxes and other DNA motifs, such as “CAT” or “SP1” boxes.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “functionally linked” and “operably linked” are used interchangeably and refer to a functional relationship between two or more DNA segments, in particular gene sequences to be expressed and those sequences controlling their expression. For example, a promoter/enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

“Conservatively modified variants” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

A conservative substitution (also called conservative replacement or conservative mutation) may include substitution such as basic for basic, acidic for acidic, polar for polar, etc. The sets of amino acids thus derived are likely to be conserved for structural reasons. These sets can be described in the form of a Venn diagram (Livingstone C. D. and Barton G. J., “Protein sequence alignments: a strategy for the hierarchical analysis of residue conservation”, Comput. Appl. Biosci. 1993, 9, 745-756; Taylor W. R., “The classification of amino acid conservation”, J. Theor. Biol. 1986, 119, 205-218), which is incorporated herein by reference.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. The invention provides polypeptides that are substantially identical to the polypeptides, respectively, exemplified herein, as well as uses thereof including, but not limited to, use for treating or preventing neurological diseases or disorders, e.g., neurodegenerative diseases or disorders, and/or treating SCI. Optionally, the identity exists over a region that is at least about 50 nucleotides in length, or more preferably over a region that is 100 to 500 or 1000 or more nucleotides in length, or the entire length of the reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math., 1970, 2:482c, by the homology alignment algorithm of Needleman and Wunsch, J. Mol. Biol., 1970, 48:443, by the search for similarity method of Pearson and Lipman, Proc. Nat'l. Acad. Sci. USA, 1988, 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology, 1995, supplement).

Two examples of algorithms that are suitable for determining percent sequence identity and sequence similarity are the BLAST and BLAST 2.0 algorithms, which are described in Altschul et al., Nuc. Acids Res., 1977, 25, 3389-3402; and Altschul et al., J. Mol. Biol., 1990, 215, 403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA, 1993, 90, 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). In various embodiments, nucleic acids are isolated when purified away from other cellular components or other contaminants (e.g., other nucleic acids or proteins present in the cell) by standard techniques including, including alkaline/SDS treatment, CsCl banding, column chromatography, agarose gel electrophoresis and others well-known in the art. See e.g., F. Ausubel, et al., ed., Current Protocols in Molecular Biology, 1987, Greene Publishing and Wiley Interscience, New York. In various embodiments, a nucleic acid is, for example, DNA or RNA and may or may not contain intronic sequences. In a preferred embodiment, the nucleic acid is a cDNA molecule.

As used herein “pharmaceutically acceptable carrier” encompasses any of the standard pharmaceutical carriers, such as a phosphate buffered saline solution, water and emulsions such as an oil/water or water/oil emulsion, and various types of wetting agents.

The term “viral vector” or “viral expression vector” as used herein refers to a nucleic acid vector that includes at least one element of a virus genome and may be packaged into a viral particle. In the context of the present invention, the term “viral vector” has to be understood broadly as including nucleic acid vector (e.g., DNA viral vector) as well as viral particles generated thereof. In this application, the viral expression vector includes adeno-associated virus (AAV) vector and herpes simplex virus (HSV) vector.

The term “AAV” refers to the Adeno-Associated Virus itself or to derivatives thereof including recombinant AAV vector particles. Furthermore, as used herein, the term “AAV” includes many different serotypes, which have been isolated from both human and non-human primate samples. Preferred AAV serotypes are the human serotypes, more preferably human AAV of serotypes 2, 5 and 9, most preferably human AAV of serotype 5, which is the serotype displaying the highest level of neurotropism.

The term “herpes simplex virus (HSV)” is a complex, non-integrating DNA virus capable of infecting a very wide range of human and animal cells. HSV encompasses two serotypes, herpes simplex virus type 1 (HSV-1) and herpes simplex virus type 2 (HSV-2).

As used herein, the term “replication competent virus” refers to a virus which contains all the information within its genome to allow it to replicate within a cell.

As used herein the term “replication incompetent viral vector” or “defective viral vector” shall refer to viral vectors that are missing genes or parts of genes necessary to successfully complete the viral life cycle in order to replicate.

The expression “recombinant DNA” as used herein describes a nucleic acid molecule, i.e., a polynucleotide of genomic, cDNA, viral, semisynthetic, and/or synthetic origin, which, by virtue of its origin or manipulation, is not associated with all or a portion of the polynucleotide with which it is associated in nature. The term “recombinant” as used with respect to virus means a virus carrying a recombinant genome or a genome that has been manipulated to introduce mutations, deletions or one or more heterologous polynucleotides, including genes. The term “recombinant” as used with respect to a protein or polypeptide, means a polypeptide produced by expression of a recombinant nuclei acid. The term “recombinant” as used with respect to a host cell means a recombinant vector that carries recombinant DNA within the host cell or a cell that contains recombinant DNA inserted in its genome. The term “infection” refers to the ability of a viral vector to enter a host cell or organ or subject, or the ability of a gene product of the viral vector to enter a host cell.

### HSV-1 Vector

Provided in this application is a modified HSV-1 vector comprising a mutation, wherein such mutation allows for the production of the HSV-1 vector in suspension cell lines.

This application also provides a modified HSV-1 vector comprising an inactivating deletion of US8 (gE) gene.

In some embodiments, the parental HSV-1 vector is a wild-type HSV-1 before the introduction of the mutation into its genome. The genome of a wild-type HSV-1 has a size of approximately 153-kbp, and is composed of two unique segments, UL and US, each flanked by inverted repeats that encode critical diploid genes. It contains about 90 protein encoding genes and more than 12 microRNA. In a preferred embodiment, before the introduction of the mutation into its genome, the parental HSV-1 vector is the human HSV-1 strain F comprising the genome of GenBank Accession No. GU734771.1. In some embodiments, “modified” and “mutant” used in the expressions of “modified HSV-1 vector” and “mutant HSV-1 vector” are interchangeable.

In some embodiments, the mutation that allows for the production of the modified HSV-1 vector in suspension cell lines is a single nucleotide substitution introduced to the coding sequence of US2 (SEQ ID NO: 1) within the genome of HSV-1. The HSV US2 gene is non-essential for viral replication in cell culture and predicted to encode a 291-aa protein of 33 kDa.

In a preferable embodiment, the mutation introduced to the coding sequence of US2 (SEQ ID NO: 1) within the genome of HSV-1 that allows for the production of the modified HSV-1 vector in suspension cell lines results in a glycine-276 to valine (G276V) amino acid substitution, which position is numbered relative to the position in the wild-type US2 gene. In another preferable embodiment, the coding sequence of US2 in the modified HSV-1 vector comprises the nucleic acid sequence of SEQ ID NO: 2. In yet another embodiment, the coding sequence of US2 in the modified HSV-1 vector consists of the nucleic acid sequence of SEQ ID NO: 2.

In some embodiments, the mutation that allows for the production of the modified HSV-1 vector in suspension cell lines is a single nucleotide substitution introduced to the coding sequence of US8 (SEQ ID NO: 3) within the genome of HSV-1. The HSV-1 US8 gene (also called gE gene) is non-essential for viral replication in cell culture and encodes a 552-aa protein called glycoprotein E (AKA gE). gE and gI (glycoprotein I, expressed by HSV-1 US7 gene) tend to form heterodimers. In epithelial cells, the heterodimer gE/gI is required for the cell-to-cell spread of the virus, by sorting nascent virions to cell junctions. Once the virus reaches the cell junctions, virus particles can spread to adjacent cells extremely rapidly through interactions with cellular receptors that accumulate at these junctions (See Johnson et al., Journal of Virology, 2001, Volume 75, Issue 2, 821-833. https://doi.org/10.1128/JVI.75.2.821-833.2001). In neuronal cells, gE/gI is essential for the anterograde spread of the infection throughout the host nervous system. Together with US9 protein, the heterodimer gE/gI is involved in the sorting and transport of viral structural components toward axon tips (See Snyder et al., Journal of Virology, 2008, Volume 82, Issue 21, 10613-10624. https://doi.org/10.1128/JVI.01241-08).

In a preferable embodiment, the mutation introduced to the coding sequence of US8 (SEQ ID NO: 3) within the genome of HSV-1 that allows for the production of the modified HSV-1 vector in suspension cell lines results in a premature STOP codon at amino acid position 116 (W116STOP). In another embodiment, the coding sequence of US8 in the modified HSV-1 vector comprises the nucleic acid sequence of SEQ ID NO: 4. In yet another embodiment, the coding sequence of US8 in the modified HSV-1 vector consists of the nucleic acid sequence of SEQ ID NO: 4.

The suspension cell lines contemplated include, without limitation, CHO, HeLa, H-9, Jurkat, C6/36, High Five, S2, Sf21, Sf9, PC-1, and HEK293. Preferably, the suspension cell line contemplated is HEK293.

This application also provides a modified HSV-1 vector comprising an inactivating deletion of US8 (gE) gene. The inactivating deletion can be a deletion within, or of, the entire coding sequence of the US8 (gE) gene or alternatively including the promoter or other regulatory sequences of such gene. In one aspect, the inactivating deletion can be a complete deletion of the coding sequence of the US8 (gE) gene, such that the virus genome does not contain nucleic acid sequences of the US8 (gE) gene. As used herein, an “inactivating deletion” of the US8 gene is any deletion that results in the absence of the US8 protein on the surface of the HSV-1 virus.

In some embodiments, the inactivating deletion comprises the single nucleotide substitution in the coding sequence of US8 as described herein.

As used herein, “US8”, “US8 (gE)”, and “gE” are used interchangeably to refer to the envelope glycoprotein E gene or protein as determined by the context in which it is used.

The gE protein is a viral surface glycoprotein embedded in its envelope. The N-terminal part constitutes the external part of the protein with the transmembrane domain being located between residues 420 to 440. Without wishing to be bound by any particular theory, it is believed that the mutation leading to an early stop of the US8 (gE) protein synthesis (115 aa vs. 552 aa for the native protein), it is likely that this mutation results in the complete absence of the protein within the mutant virus stocks.

### Attenuated HSV-1 Vector

In embodiments, the modified HSV-1 vector as described herein is an attenuated HSV-1 vector.

Provided in this application is also an attenuated HSV-1 vector comprising a mutation as described herein. The mutation allows for the production of the attenuated HSV-1 vector in suspension cell lines.

Also provided in this application is an attenuated HSV-1 vector comprising an inactivating deletion of the US8 gene. The inactivating deletion allows for the production of the attenuated HSV-1 vector in suspension cell lines.

As used herein, the terms “attenuated” and “replication competent” are interchangeable and both describe a vector derived from an attenuated virus, of which nonessential genes are either mutated or deleted. The removal of one or more nonessential genes may reduce pathogenicity without requiring a cell line to complement growth. Replication-competent HSV-1 vectors with mutations in genes that affect viral replication, neuropathogenicity, and immune evasiveness have been developed and tested for their safety and efficacy in a variety of mouse models.

As used herein, the qualifier “essential” in the expressions “essential genes” or “non-essential genes”, means that the given gene is essential (or not) for achieving multiplication and packaging of the virus genome, thus generating infectious progeny virus particles. HSV-1 essential genes include UL1, UL5-UL9, UL12, UL14, UL15, UL17-UL19, UL22, UL25-UL38, UL42, UL48, UL49, UL52-UL54, US6, ICP4 (2 copies). HSV-1 non-essential genes include ICP34.5 (2 copies), ICP0 (2 copies), LAT (2 copies), UL2-UL4, UL10, UL11, UL13, UL16, UL20, UL21, UL23, UL24, UL39, UL40, UL41, UL43-UL47, UL50, UL51, UL55, UL56, US1-US5, US7-US12.

In any embodiment wherein nonessential genes are either mutated or deleted to arrive at the attenuated HSV-1 vector as described herein, at least one of the two nonessential genes US2 and US8 is preserved in the attenuated HSV-1 genome, for the introduction of the mutation as described herein.

In some embodiments, the mutation that allows for the production of the attenuated HSV-1 vector in suspension cell lines is a single nucleotide substitution introduced to the coding sequence of US2 (SEQ ID NO: 1) within the genome of attenuated HSV-1.

In a preferable embodiment, the mutation introduced to the coding sequence of US2 (SEQ ID NO: 1) within the genome of attenuated HSV-1 that allows for the production of the attenuated HSV-1 vector in suspension cell lines results in a glycine-276 to valine (G276V) amino acid substitution, which position is numbered relative to the position in the wild-type US2 gene. In another preferable embodiment, the coding sequence of US2 in the attenuated HSV-1 vector comprises the nucleic acid sequence of SEQ ID NO: 2. In yet another embodiment, the coding sequence of US2 in the attenuated HSV-1 vector consists of the nucleic acid sequence of SEQ ID NO: 2.

In some embodiments, the mutation that allows for the production of the attenuated HSV-1 vector in suspension cell lines is a single nucleotide substitution introduced to the coding sequence of US8 (SEQ ID NO: 3) within the genome of HSV-1.

In a preferable embodiment, the mutation introduced to the coding sequence of US8 (SEQ ID NO: 3) within the genome of attenuated HSV-1 that allows for the production of the attenuated HSV-1 vector in suspension cell lines results in a premature STOP codon at amino acid position 116 (W116STOP). In another embodiment, the coding sequence of US8 in the attenuated HSV-1 vector comprises the nucleic acid sequence of SEQ ID NO: 4. In yet another embodiment, the coding sequence of US8 in the attenuated HSV-1 vector consists of the nucleic acid sequence of SEQ ID NO: 4.

In some embodiments, the inactivating deletion of the US8 gene allows for the production of the attenuated HSV-1 vector in suspension cell lines.

The attenuated HSV-1 vectors contemplated herein include any attenuated HSV-1 vectors being clinically available, being developed and tested, or under development. The attenuated HSV-1 vectors also include any attenuated HSV-1 vectors that do not exist at the time of this invention but will be developed in the future.

In some embodiments, the attenuated HSV-1 vectors contemplated herein may be oncolytic HSV-1 vectors that preferentially infect and kill tumor cells. Nonlimiting examples of oncolytic HSV-1 vectors that can be used in this application include hrR3 (ICP6-defective), R3616 (γ34.5-deleted), G207 (γ34.5-deleted and ICP6-defective), HSV1716 (γ34.5-deleted), T-VEC (Talimogene laherparepvec, γ34.5-deleted and ICP-deleted). The oncolytic HSV-1 vectors that can be used in this application may be selected from these described in Peters. Et al., Molecular Therapy—Oncolytics, 2015, 2, 15010. https://doi.org/10.1038/mto.2015.10.

The suspension cell lines contemplated include, without limitation, CHO, HeLa, H-9, Jurkat, C6/36, High Five, S2, Sf21, Sf9, PC-1, and HEK293. Preferably, the suspension cell line contemplated is HEK293.

### Defective HSV-1 Vector

In embodiments, the modified HSV-1 vector as described herein is a defective HSV-1 vector.

Provided in this application is also a defective HSV-1 vector comprising a mutation as described herein. The mutation allows for the production of the defective HSV-1 vector in suspension cell lines.

Also provided in this application is a defective HSV-1 vector comprising an inactivating deletion of the US8 gene as described herein. The inactivating deletion allows for the production of the defective HSV-1 vector in suspension cell lines.

As used herein, the terms “defective” and “non-replicative” in the expressions “defective HSV-1” and “non-replicative HSV-1” are interchangeable and both refer to a vector derived from a defective virus, of which genes essential for viral replication are either mutated or deleted. These deletions have substantially reduced the cytotoxicity of the viral vectors by preventing early and late viral gene expression and, together with other deletions involving “nonessential” genes, have also created space to introduce distinct and independently regulated expression cassettes for different transgenes. Therapeutic effects in gene therapy applications requiring simultaneous and synergic expression of multiple gene products can be easily achievable with these defective vectors.

The defective HSV-1 vector contemplated may be a helper-independent defective HSV-1 vector.

In some embodiments, the genome of the helper-independent defective HSV-1 vectors in this application may comprise at least complete deletions of the genes coding for two essential proteins ICP4 and ICP27. The ICP4 gene is present in two copies, located in the inverted repeated sequences known as c and c′ of the virus genome, Internal Repeat Short and Terminal Repeat Short, respectively, and both copies of this gene are deleted. The gene encoding ICP27 (UL54) is located in the unique long (UL) sequence of the virus genome. This defective HSV-1 vector can further lack other genes, coding for non-essential proteins, such as ICP34.5, UL55, UL56 and/or UL41 proteins, and carries the expression cassette(s) embedded into the vector genome (preferably, the LAT (Latent Associated Transcripts) regions of the vector genome).

In some embodiments, the defective HSV-1 vector further lacks one copy of the ICP0 gene. In a preferred embodiment, the one copy of the IPC0 gene is removed among the LAT, ICP0, UL34.5 cluster from the IRL (Internal Repeat Long) region of the HSV vector.

In some embodiments, the vector according to the invention is a defective vector carrying the expression cassettes described herein driven by promoters as described in other parts of this document.

In any embodiment wherein essential and/or nonessential genes are either mutated or deleted to arrive at the defective HSV-1 vector as described herein, at least one of the two nonessential genes US2 and US8 is preserved in the defective HSV-1 genome, for the introduction of the mutation as described herein.

In some embodiments, the mutation that allows for the production of the defective HSV-1 vector in suspension cell lines is a single nucleotide substitution introduced to the coding sequence of US2 (SEQ ID NO: 1) within the genome of defective HSV-1.

In a preferable embodiment, the mutation introduced to the coding sequence of US2 (SEQ ID NO: 1) within the genome of defective HSV-1 that allows for the production of the defective HSV-1 vector in suspension cell lines results in a glycine-276 to valine (G276V) amino acid substitution, which position is numbered relative to the position in the wild-type US2 gene.

In another preferable embodiment, the coding sequence of US2 in the defective HSV-1 vector comprises the nucleic acid sequence of SEQ ID NO: 2. In yet another embodiment, the coding sequence of US2 in the defective HSV-1 vector consists of the nucleic acid sequence of SEQ ID NO: 2.

In some embodiments, the mutation that allows for the production of the defective HSV-1 vector in suspension cell lines is a single nucleotide substitution introduced to the coding sequence of US8 (SEQ ID NO: 3) within the genome of HSV-1.

In a preferable embodiment, the mutation introduced to the coding sequence of US8 (SEQ ID NO: 3) within the genome of defective HSV-1 that allows for the production of the defective HSV-1 vector in suspension cell lines results in a premature STOP codon at amino acid position 116 (W116STOP). In another embodiment, the coding sequence of US8 in the defective HSV-1 vector comprises the nucleic acid sequence of SEQ ID NO: 4. In yet another embodiment, the coding sequence of US8 in the defective HSV-1 vector consists of the nucleic acid sequence of SEQ ID NO: 4.

In some embodiments, the inactivating deletion of the US8 gene allows for the production of the defective HSV-1 vector in suspension cell lines.

The defective HSV-1 vectors contemplated herein include any defective HSV-1 vectors being clinically available, being developed and tested, or under development. The defective HSV-1 vectors also include any defective HSV-1 vectors that do not exist at the time of this invention but will be developed in the future.

Nonlimiting examples of the defective HSV-1 vectors can be used as described in this application include HSV-1 d106S, KOS strain 5d11.2, strain 17 D30EBA, KOS strain d120, and NP2. The defective HSV-1 vectors used in this application can be selected from these described in the US Patent U.S. Ser. No. 11/414,666B2, these described in the U.S. Application No. 63/284,176, or these described in the French Application FR2212771. In some embodiments, the defective HSV-1 vector used in this application may be a “pre-HSV-1 vector” described in the U.S. Application No. 63/284,176, or in the French Application FR2212771, wherein non-essential genes, essential genes, or combinations thereof have been deleted to arrive at a genome comprising less than 130 kbp and greater than 75 kbp.

The suspension cell lines contemplated include, without limitation, CHO, HeLa, H-9, Jurkat, C6/36, High Five, S2, Sf21, Sf9, PC-1, and HEK293. Preferably, the suspension cell line contemplated is HEK293.

The defective HSV-1 vector contemplated may be a helper-dependent defective HSV-1 vector, i.e., an HSV-1 amplicon vector.

By “amplicon or amplicon vector” it is meant a helper-dependent defective vector, the genome of which lacks most or all HSV genes coding for virus proteins. The genome of amplicon vectors is a concatemeric DNA composed of multiple copies in tandem of a plasmid, known as the amplicon plasmid, that carries one origin of DNA replication and one packaging signal from HSV-1 genome, in addition to transgenic DNA (i.e., expression cassettes) of interest. The term “helper-dependent” as used herein refers to a viral vector that is dependent on the assistance of a helper virus in order to replicate.

In any embodiments wherein the defective HSV-1 vector is an HSV-1 amplicon vector, a helper virus-dependent packaging system is used for the production of HSV-1 amplicon vector, wherein the helper virus-dependent packaging system comprises a helper HSV-1 vector, and wherein the mutation as described herein is introduced to the genome of the helper HSV-1 vector. In some embodiments, the helper HSV-1 vector comprises the nucleic acid sequence of SEQ ID NO: 2 in the US2 coding sequence region. In some embodiments, the helper HSV-1 vector comprises the nucleic acid sequence of SEQ ID NO: 4 in the US8 (gE) coding sequence region. In some embodiments, the helper HSV-1 vector comprises the nucleic acid sequence of SEQ ID NO: 2 in the US2 coding sequence region and the nucleic acid sequence of SEQ ID NO: 4 in the US8 (gE) coding sequence region.

In some embodiments, the helper HSV-1 vector comprises an inactivating deletion within the US8 (gE) gene.

In some embodiments, the HSV-1 amplicon vector may not comprise a nucleic acid sequence comprising the mutation as described herein but may comprise a protein comprising the mutation as described here, preferably on the surface of the HSV-1 amplicon vector envelope or in the tegument of the HSV-1 amplicon. In some embodiments, the HSV-1 amplicon vector may comprise a mutant US2 protein, expressed by SEQ ID NO: 2, that comprise a G276V mutation relative to the wild US2 protein. In some embodiments, the HSV-1 amplicon vector may comprise a mutant US8 protein (gE), expressed by SEQ ID NO: 4, that comprise a W116STOP mutation relative to the wild US8 protein (gE). In some embodiments, the HSV-1 amplicon vector may comprise a mutant US2 protein, expressed by SEQ ID NO: 2, that comprise a G276V mutation relative to the wild US2 protein; and a mutant US8 protein (gE), expressed by SEQ ID NO: 4, that comprise a W116STOP mutation relative to the wild US8 protein (gE).

In some embodiments, the HSV-1 amplicon vector may comprise an inactivating deletion in the US8 (gE) protein. In some embodiments, the modified HSV-1 vector is an HSV-1 amplicon vector that comprise a modified US8 (gE) protein or lacks the US8 (gE) protein. In embodiments, the inactivating deletion of the US8 gene in the helper virus can result in a non-functional US8 protein in the HSV-1 amplicon vector. In embodiments, the inactivating deletion of the US8 gene in the helper virus can result in the lack of the US8 protein in the HSV-1 amplicon vector.

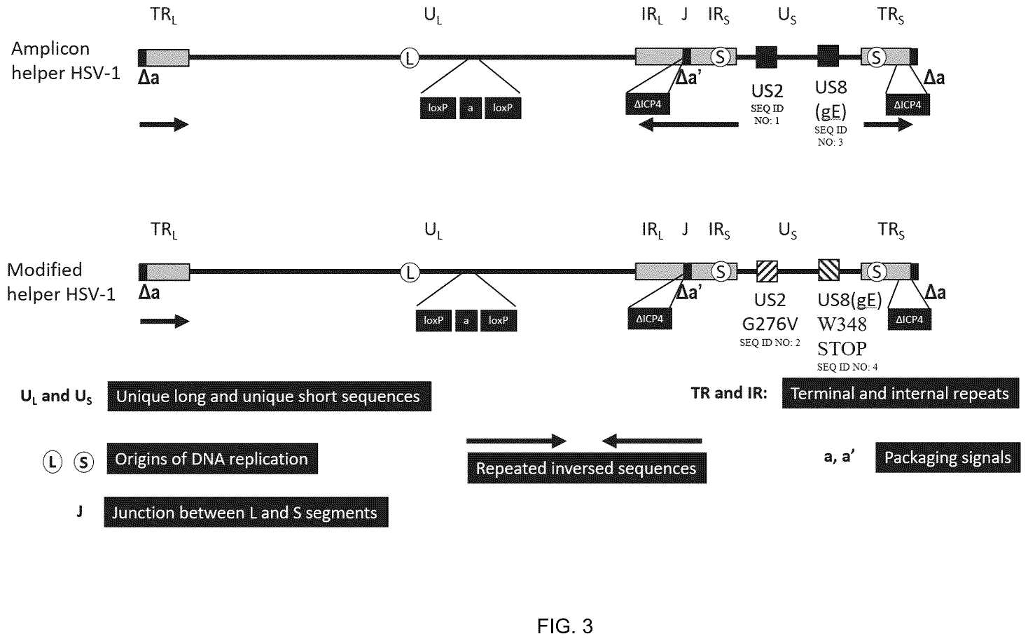

In any embodiments wherein the defective HSV-1 vector is an HSV-1 amplicon vector, the mutation as described herein is introduced to the genome of a helper HSV-1 vector used in a helper virus-dependent packaging system for the production of the HSV-1 amplicon vector. As a nonlimiting example, Zaupa et al. described an improved packaging system that uses Cre-1oxP site-specific recombination to delete the packaging signals of the genome of defective helper HSV-1 vector and therefore generates high-level noncytotoxic HSV-1 amplicon vectors. (See Human Gene Therapy. July 2003, 1049-1063. http://doi.org/10.1089/104303403322124774.) As depicted in FIG. 3, the helper HSV-1 vector of Zaupa et al. is used herein as an exemplary parental helper HSV-1 vector for introducing the mutations of this present application.

### Additional Genetic Modifications

In some embodiments, the modified HSV-1 vectors as described herein (i.e., the HSV-1 vector, the attenuated HSV-1 vector, and the defective HSV-1 vector, each with a mutation as described herein), may further comprise additional genetic modifications, such as insertions of one or more transgenes of interest or deletions of one or more nucleotide sequences.

In some embodiments, the modified HSV-1 vectors may further comprise a genome comprising exogenous expression cassette(s). The expression cassette may comprise at least one nucleic acid sequence encoding a gene product (i.e., at least one transgene of interest).

Preferably, the genome comprising exogenous expression cassette(s) can be introduced into a LAT locus, which is a repeated locus that is contained in the inverted repeated sequences known as b and b′ of the virus genome. The b and b′ sequences of the virus genome are also known as TRL (Terminal Repeat Long) and IRL (Internal Repeat Long), respectively. In some embodiments, the virus genome contains both LAT regions, one in the TRL and the other in the IRL. In some embodiments one of the LAT regions, either in the TRL or in the IRL has been deleted. In some embodiments, when the vector genome contains the two LAT loci, the genome comprising exogenous expression cassette(s) can be introduced into both loci, in the TRL region and in the IRL region. In some embodiments, when the LAT loci in the IRL region is deleted, the genome comprising exogenous expression cassette(s) can be introduced into the LAT locus in the TRL region only. In some embodiments, when the LAT loci in the TRL region is deleted, the genome comprising exogenous expression cassette(s) can be introduced into the LAT locus in the IRL region only.

The LAT locus includes an upstream DNA insulator (INS) sequence, the Latency Associated Promoter (LAP), a region conferring Long-Term Expression (LTE) and a downstream DNA insulator (INS). In some embodiments, the genome comprising exogenous expression cassette(s) is introduced either between the Latency Associated Promoter (LAP) and the Long-Term Expression (LTE) region, or between the LTE region and the DNA insulator (INS) sequence present downstream of the LTE.

In a preferable embodiment, a genome comprising exogenous expression cassette(s) is placed either between the Latency Associated Promoter (LAP) and the Long-Term Expression (LTE) region (site 1), or between the LTE region and the DNA insulator (INS) sequence present downstream of the LTE (site 2). In some embodiments, one or more HSV-1 non-essential genes is introduced into the modified HSV-1 vectors.

Various transgenes of interest can be introduced into the exogenous expression cassette(s) in the modified HSV-1 vectors as described in this application. The modified HSV-1 vector as described may be used to express any viral, bacterial, or cancer gene products. For example, the transgene of interest may encode a gene product capable of inhibiting/silencing neurotransmission (e.g., wild-type or modified light chain botulinum toxins, antisense RNA (AS-RNA) targeting SNARE proteins, GAD67, GAD65, RIPs, and/or NTRs). For example, the transgene of interest may encode a gene product capable of inducing protective immunity (e.g., the SARS-CoV-2 (COVID-19) spike protein).

In some embodiments, any modified HSV-1 vector as described herein may comprise both a G276V mutation relative to the wild-type coding sequence of US2 and a W116STOP mutation relative to the wild-type coding sequence of US8. In some embodiments, the HSV-1 vector, the attenuated HSV-1 vector, and the defective HSV-1 vector, each with a mutation as described herein, may comprise both a US2 gene having a nucleic acid sequence of SEQ ID NO: 2, and a US8 gene having a nucleic acid sequence of SEQ ID NO: 4.

In some embodiments, any modified HSV-1 vector as described herein may comprise a W116STOP mutation relative to the wild-type coding sequence of US8. In some embodiments, the HSV-1 vector, the attenuated HSV-1 vector, and the defective HSV-1 vector, each with a mutation as described herein, may comprise a US8 gene having a nucleic acid sequence of SEQ ID NO: 4.

In some embodiments, any modified HSV-1 vector as described herein may comprise an inactivating deletion of the US8 gene. In some embodiments, the HSV-1 vector, the attenuated HSV-1 vector, and the defective HSV-1 vector, each with a mutation as described herein, may comprise an inactivating deletion in the US8 gene.

### Pharmaceutical Composition

The modified HSV-1 vectors as described in this application can be administered in a pharmaceutical composition which may comprise a pharmaceutically acceptable carrier. The carrier of the composition can be any suitable carrier for the vector. The carrier typically will be liquid, but also can be solid, or a combination of liquid and solid components. The carrier desirably is a pharmaceutically acceptable (e.g., a physiologically or pharmacologically acceptable) carrier (e.g., excipient or diluent). The composition can further comprise any other suitable components, especially for enhancing the stability of the composition and/or its end-use. Accordingly, there is a wide variety of suitable formulations of the composition of the viral vector. The following formulations and methods are merely exemplary and are in no way limiting.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described.

In addition, the composition can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the viral vector and physiological distress. Immune system suppressors can be administered with the composition to reduce any immune response to the vector itself or associated with a disorder. Alternatively, immune enhancers can be included in the composition to upregulate the body's natural defenses against disease. Antibiotics, i.e., microbicides and fungicides, can be present to reduce the risk of infection associated with gene transfer procedures and other disorders.

### Method of Manufacturing HSV-1 Vectors in Suspension Cell Lines

Provided in this application is also a method of producing a modified HSV-1 vector in a suspension cell line, wherein the method comprises infecting a suspension cell line with a modified HSV-1 vector as described herein; and culturing the infected cells.

As described herein, the modified HSV-1 vector comprises a mutation selected from a single nucleotide substitution introduced to the coding sequence of US2 and a single nucleotide substitution introduced to the coding sequence of US8. In an embodiment, the single nucleotide substitution introduced to the coding sequence of US2 results in a G276V amino acid substitution, which position is numbered relative to the position in wild-type US2 gene (SEQ ID NO: 1). In another embodiment, the single nucleotide substitution introduced to the coding sequence of US8 results in a premature STOP codon (W116STOP), which position is numbered relative to the position in the wild-type US8 gene (SEQ ID NO: 3).

As described herein, the modified HSV-1 vector comprises a mutation selected from a single nucleotide substitution introduced to the coding sequence of US8. In an embodiment, the single nucleotide substitution introduced to nucleotide position 348 of the coding sequence of US8 results in a premature STOP codon (W116STOP), which nucleotide position is numbered relative to the position in the wild-type US8 gene (SEQ ID NO: 3).

As described herein, the modified HSV-1 vector comprises an inactivating deletion of the coding sequence of US8.

The parental HSV-1 vector relative to the modified HSV-1 vector can be selected from a wild-type HSV-1 vector (preferably the human HSV-1 strain F comprising the genome of GenBank Accession No. GU734771.1), a recombinant HSV-1 vector, an attenuated HSV-1 vector, a non-replicative HSV-1 vector, a defective and helper independent HSV-1 vector.

Provided in this application is also a method of producing a modified HSV-1 amplicon vector in a suspension cell line, wherein the method comprises infecting a suspension cell line with a helper virus-dependent packaging system that comprises an HSV-1 amplicon vector or an HSV-1 amplicon plasmid, and a modified helper HSV-1 vector comprising the mutation as described herein; and culturing the infected cells. In some embodiments, the resultant HSV-1 amplicon vector produced by the method as described above may comprise a mutant US2 protein, expressed by SEQ ID NO: 2, that comprises a G276V mutation relative to the wild US2 protein. In some embodiments, the resultant HSV-1 amplicon vector produced by the method as described above may comprise a mutant US8 protein (gE), expressed by SEQ ID NO: 4, that comprises a W116STOP mutation relative to the wild US8 protein (gE). In some embodiments, the resultant HSV-1 amplicon vector produced by the method as described above may comprise a mutant US2 protein, expressed by SEQ ID NO: 2, that comprises a G276V mutation relative to the wild US2 protein; and a mutant US8 protein (gE), expressed by SEQ ID NO: 4, that comprises a W116STOP mutation relative to the wild US8 protein (gE). In a preferred embodiment, the mutant US2 protein is present in the tegument of the HSV-1 amplicon.

In some embodiments, the resultant HSV-1 amplicon vector produced by the method as described above may lack a US8 protein, as a result of an inactivating deletion within the US8 (gE) gene in the helper virus.

The parental HSV-1 vector contemplated can be or be derived from any HSV-1 strains as described herein. The parental HSV-1 vector includes any HSV-1 vectors being clinically available, being developed and tested, or under development. The parental HSV-1 vector includes any HSV-1 vectors that do not exist at the time of this invention but will be developed in the future. As used herein, a modified HSV-1 vector is a parental HSV-1 vector comprising one or both of the mutations described herein.

Provided in this application is also a method of producing an HSV-1 vector in suspension cell lines, wherein the HSV-1 vector is modified and comprises a US2 gene comprising a nucleic acid sequence of SEQ ID NO: 2, and/or a US8 gene comprising a nucleic acid sequence of SEQ ID NO: 4. The HSV-1 vector is selected from a wild-type HSV-1 vector such as human HSV-1 strain F, a replication competent HSV-1 vector, a defective helper-independent HSV-1 vector, and an HSV-1 amplicon vector.

Provided in this application is also a method of producing an HSV-1 vector in suspension cell lines, wherein the HSV-1 vector is modified and comprises an inactivating deletion of the US8 gene. The HSV-1 vector is selected from a wild-type HSV-1 vector such as human HSV-1 strain F, a replication competent HSV-1 vector, a defective helper-independent HSV-1 vector, and an HSV-1 amplicon vector.

The suspension cell lines contemplated include, without limitation, CHO, HeLa, H-9, Jurkat, C6/36, High Five, S2, Sf21, Sf9, PC-1, and HEK293. Preferably, the suspension cell line contemplated is HEK293.

In some embodiments wherein the HSV-1 vectors are defective HSV-1 vectors, the suspension cell lines used in the method of this invention may simultaneously express the proteins ICP4 and ICP27.

The following examples are intended to illustrate but not limit the invention. All citations throughout the disclosure are hereby expressly incorporated by reference.

### EXAMPLES

### Example 1

### Screening HSV-1 Mutants for Suspension Cell Production

The original HSV-1 WT stock (P0) was generated from the bacterial artificial chromosome (BAC) carrying the full sequence of HSV-1 strain F via transient transfection into Vero 7b cells using transfection reagent lipofectamine 2000. The excision of the BAC element, flanked by 1oxP recombination sites, from the genome was ensured through co-transfection with a plasmid encoding for Cre recombinase. The resulting virus stock was harvested, and the titer was determined using plaque assay on Vero 7b cells.

HSV-1 WT virus stocks H1 through H10 were produced by subsequent rounds of infection of suspension HEK293F cells (ref. A35347). To obtain HSV-1 WT H1, 30×106 cells were pelleted at 300 g for 5 min, resuspended in 5 mL of LV-MAXTM Production Medium and infected with HSV-1 WT P0 at MOI of 0.05. After a 90 min incubation on a roller shaker, infected cells were transferred into a 125 mL Erlenmeyer flask (ref. 431143), topped up to 30 mL with culture medium and cultured for 96 hours at 37° C. with agitation (130 rpm, ref. 88881102). To harvest the virus, dextran sulfate and sodium chloride were added to the culture media at the final concentrations of 0.33 M and 73 g/mL, respectively, together with DNase (ref D4527-20KU) followed by incubation for 4 hours at 37° C. Cell debris were pelleted by centrifugation at 2,000 g for 5 min at 4° C. and discarded, the supernatant was filtered through 0.45 um PES filters (ref. 257201). Viruses were then pelleted at 21,000 g for one hour at 4° C. and resuspended in 1 mL of PBS. The resulting HSV-1 H1 stock was then titrated by plaque assay and used for the subsequent infection round to obtain the stock H2, etc.

The stock P1 was obtained using the method as above, except for using Vero 7b cells cultured in DMEM (ref. 41965-039) supplemented with 10% FBS (ref. 10500-064).

The purified virus stocks were then used for DNA isolation using the genomic DNA extraction kit. The isolated DNA samples were used for library preparation using Oxford Nanopore rapid barcoding kit (SQK-RBK110-96), and the sequencing was performed using flow cell FLO-MIN106 and minION Mk1B sequencing device. Basecalling was performed using MinKNOW GUI 5.3.6 at the high accuracy model, where reads shorter than 200 bp or of the overall score lower than 9 were filtered out. The resulting sequencing data was analyzed using Geneious Prime® 2022.2.2 using the “map to reference” function that utilizes the Minimap 2.24 mapping algorithm. The variants were identified using the built-in “Find variations/SNPs” function using the following parameters: Minimum variant frequency of 0.05, Maximum variant p-value of 10⁻⁶, Find variants only inside CDS, Homopolymer quality reduction of 50%.

The indel mutations resulting in frameshift as well as variations in homopolymer lengths were discarded. From the obtained variants, the ones that appeared at increasing frequency across the subsequent passages H1 to H10, while completely absent in passage P1, were selected for further validation. The two identified hits concern:
1) a single nucleotide substitution in the coding sequence of US2 that leads to the change of glycine-276 to valine, and
2) a single nucleotide substitution in the coding sequence of US8 (a.k.a glycoprotein E) that results in a formation of a premature STOP codon in position 116, thus truncating the protein (original length of US8 is 552 amino acids).

TABLE 1
Identified Mutations

| Protein | US8 | US2 |
|---|---|---|
| Genomic nucleotide position | 155932 | 148442 |
| CDS nucleotide position | 348 | 827 |
| Amino acid position | 116 | 276 |
| Amino acid change | truncation | G -> V |
| H1 [%] | <1 | <1 |
| H2 [%] | <1 | 0 |
| H3 [%] | 6.3 | <5 |
| H4 [%] | 10.8 | <5 |
| H5 [%] | 11.4 | 6.7 |
| H6 [%] | 12.5 | 6.8 |
| H7 [%] | 6.5 | <5 |
| H8 [%] | <5 | <5 |
| H9 [%] | 13.2 | 7.1 |
| H10 [%] | 10.4 | 7.7 |
| P1 [%] | 0 | 0 |

### Example 2: GE (US8) Mutation Improves HSV Manufacturing Yield in HEK293 Suspension Cell Line

To evaluate the influence of the 2 US2 and US8 mutations on production yield in HEK293 suspension cell line, 3 HSV-1 recombinant vectors were designed from HSV-1 WT:
HSV-1 mUS2: carrying only the US2_G276V mutation
HSV-1 tUS8: carrying only the US8_W116STOP mutation
HSV-1 mUS2_tUS8: carrying both US2_G276V and US8_W116STOP mutations

Each of these recombinant HSV-1 vectors were then produced in HEK293F cells (ref. A35347) in 6-well plates, at multiplicity of infection of 0.03, 1E+06 cells/mL with 2 mL/well, in technical duplicate. The produced vectors were harvested, and the titer was determined using plaque assay on Vero 7B cells at 24 h, 48 h, 72 h, and 96 h.

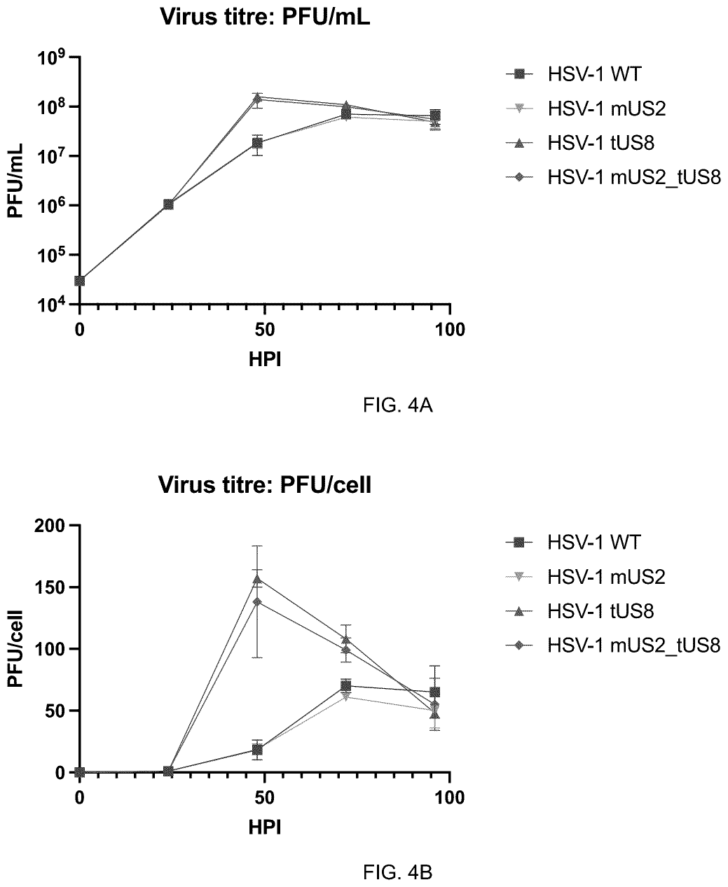

As shown in FIGS. 4A and 4B, only the truncated US8 mutation W116STOP leads to improved manufacturing yield of HSV-1 vectors in HEK293 in suspension.

### Example 3: GE (US8) Mutation Improves HSV Manufacturing Yield in HEK293 Suspension Cell Line

To further evaluate the influence of the US8 mutation on production yield in HEK293 suspension cell line, the HSV-1 WT and HSV-1 tUS8 vectors have been used for infection of HEK293F cells in Erlenmeyer flasks.

Each of these recombinant HSV-1 vectors were produced in HEK293F cells (ref. A35347) in 125 mL vented cap Erlenmeyer flasks, at multiplicity of infection of 0.05, 1E+06 cells/mL in 30 mL final, in biological triplicate. The produced vectors were harvested, and the titer was determined using plaque assay on Vero 7B cells at 24 h, 48 h, 72 h, and 96 h.

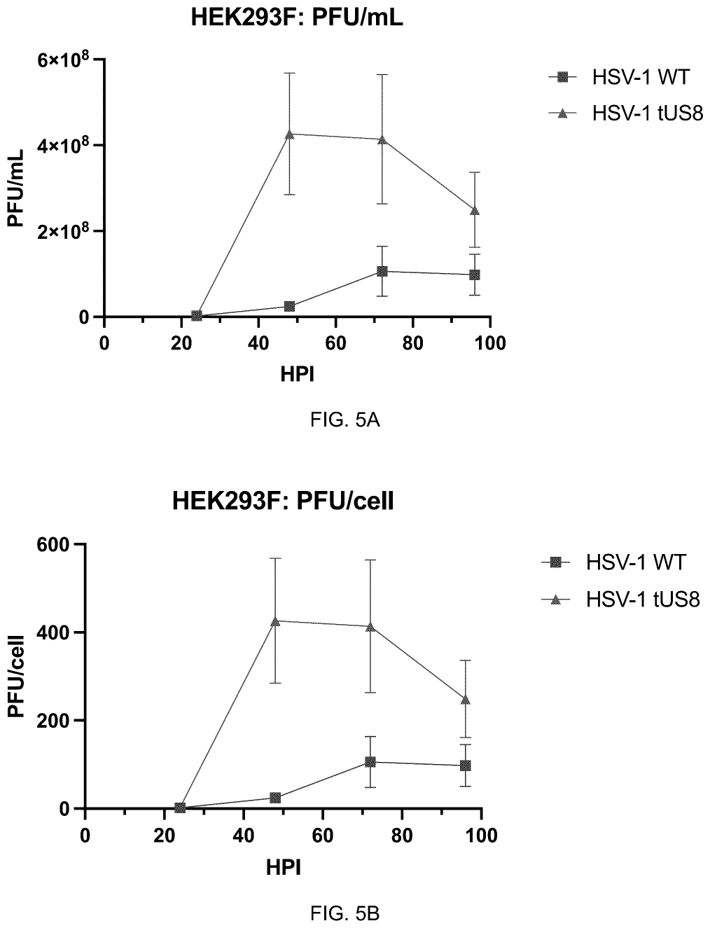

As shown in FIGS. 5A and 5B, only the truncated US8 mutation W116STOP leads to improved manufacturing yield of HSV-1 vectors in HEK293 in suspension.

### Example 4: GE (US8) Mutation Impact on Productivity is Linked to the Spread of HSV Particles

To evaluate the conditions of gain of productivity obtained from gE mutation, 2 experiments were conducted wherein HSV-1 WT and HSV-1 tUS8 were grown in 2 different cell lines:
Vero cell line in adherence; and
SH-SY5Y cell line in adherence.

Each of these recombinant HSV-1 vectors were then produced in each cell lines and the produced vectors were harvested, and the titer was determined using plaque assay on Vero 7B cells at 24 h, 48 h, 72 h, and 96 h.

For cells in suspension, the vectors were produced in 125 mL vented cap Erlenmeyer flasks, at multiplicity of infection of 0.05, at 1E+06 cells/mL in 30 mL total, in biological triplicate.

For the adherent cell lines, the viruses were produced in 24-well plates, at multiplicity of infection of 0.03, 1E+05 cells/well in technical duplicate.

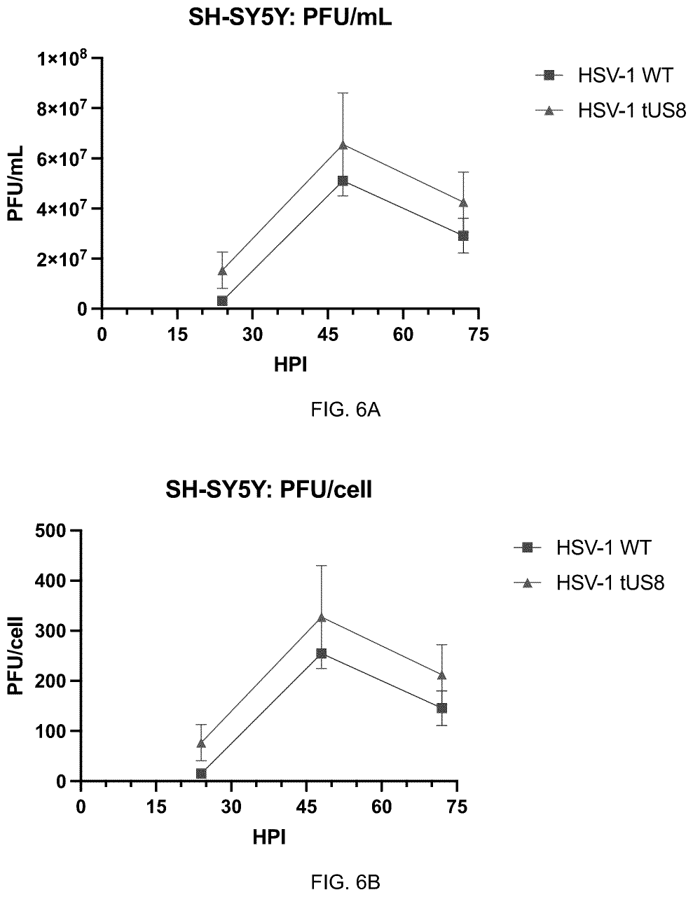

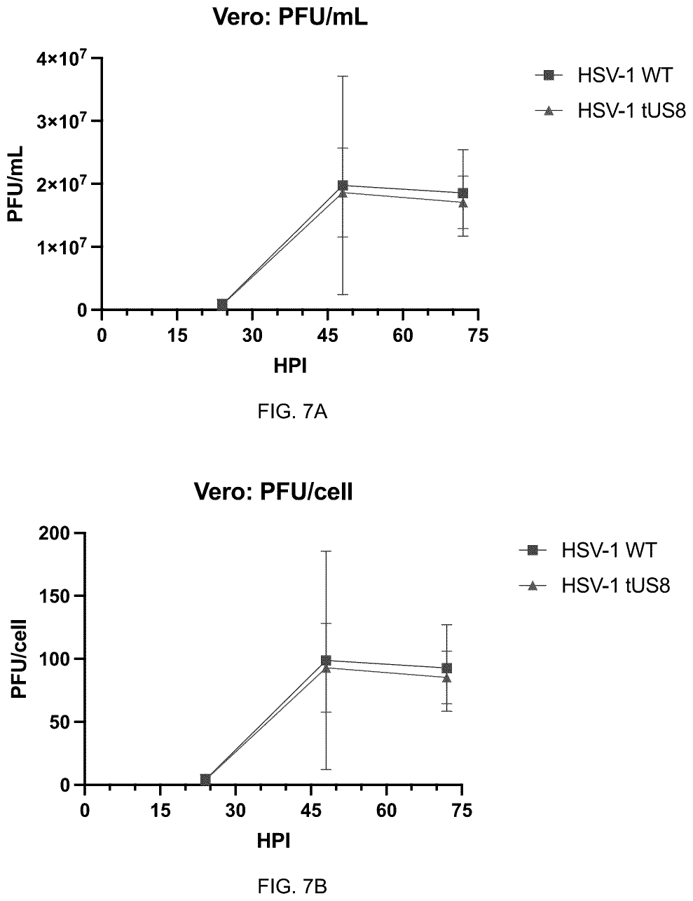

Based on the results, the improved yield of the HSV-1 tUS8 is linked to the suspension of the cells in the medium, not to the cell type (FIGS. 6 and 7). These results also demonstrate that the US8 W116 STOP mutation does not impair the replication or infectivity of the modified HSV-1 vector in other cell types.

### Example 5: GE (US8) Mutation Increase the Proportion of the HSV-1 Vectors Available in the Supernatant

Titration of produced HSV-1 particles are realized based on the retrieval of particles both in the supernatant and in the cells. From a manufacturing standpoint, the retrieval of the particles from the cells require lysate of the cells, that can be obtain by different methods known in the art, that will all lead to further increase of purification requirement to achieve high quality preparation of HSV-1 products.

The fraction of the HSV-1 tUS8 particles available in the supernatant was investigated and compared to the overall particles produced, in HEK293 cell lines (5B8 clone).

Viruses were produced as described in Example 2 except that for each collection timepoint, additional 200 μL of the culture were sampled and centrifuged at 2000 g for 5 min in addition of the 200 μL of bulk culture. Titers of the different fractions were determined by plaque assay using Vero 7B cells.

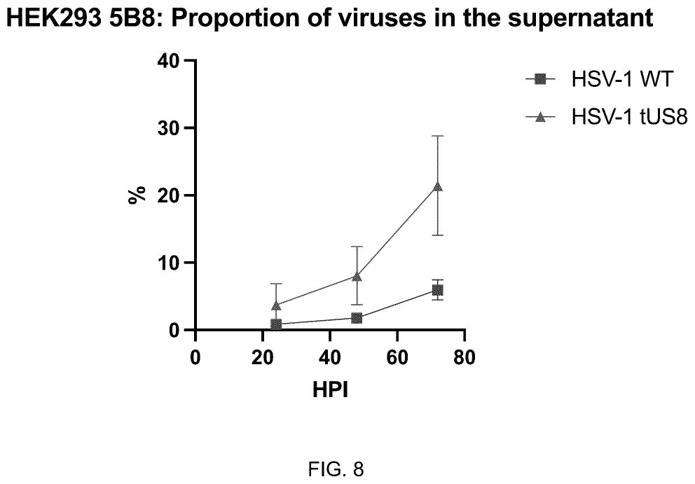

As shown in FIG. 8, the HSV-1 tUS8 allows larger proportion of the produced particles available in the supernatant.

SEQ ID NO: 1
DNA
HSV-1 US2
5′ATGGGCGTTGTTGTCGTCAACGTAATGACCCTCCTTGACCAGAACAACGCCCTGCC
CCGGACTTCCGTCGACGCAAGCCCGGCCCTGTGGAGCTTCCTGTTTAGGCAGTGCCG
CATTTTGGCATCAGAACCCCTGGGTACCCCGGTCGTCGTTCGTCCGGCCAACCTTCG
ACGGTTGGCCGAGCCGCTGATGGACTTACCCAAACCCACCCGCCCGATCGTGCGCA
CTAGGTCCTGTCGCTGCCCCCCAAACACCACCACGGGCCTGTTTGCGGAGGACAGCC
CCTTGGAGAGCACCGAGGTCGTGGACGCCGTGGCGTGCTTCCGACTGCTGCACCGA
GACCAACCCAGCCCCCCTCGCCTCTACCACTTGTGGGTGGTAGGCGCGGCGGATCTG
TGTGTGCCGTTTCTCGAATACGCCCAAAAAATCCGGCTCGGGGTAAGATTTATCGCC
ATCAAGACCCCAGACGCGTGGGTGGGAGAACCGTGGGCCGTGCCGACTCGGTTTTT
GCCCGAGTGGACCGTGGCGTGGACCCCGTTCCCCGCGGCCCCCAACCACCCCCTGG
AGACCCTGCTCAGCCGGTACGAATACCAGTACGGCGTGGTACTGCCCGGGACAAAC
GGACGGGAGCGCGATTGTATGCGCTGGCTGCGGTCCCTGATTGCTCTGCACAAACCC
CACCCAGCTACCCCAGGCCCCCTTACGACGTCCCATCCGGTGCGGCGTCCGTGTTGT
GCGTGTATGGGCATGCCCGAGATCCCAGACGAGCAACCCACATCACCGGGCCGTGG
TCCGCAAGAAACGGACCCTCTGATCGCCGTTCGCGGCGAACGGCCCAGACTTCCTCA
CATCTGCTATCCGGTTACCACCCTGTAG3′
SEQ ID NO: 2
DNA
Artificial sequence HSV-1 US2_G276V
5′ATGGGCGTTGTTGTCGTCAACGTAATGACCCTCCTTGACCAGAACAACGCCCTGCC
CCGGACTTCCGTCGACGCAAGCCCGGCCCTGTGGAGCTTCCTGTTTAGGCAGTGCCG
CATTTTGGCATCAGAACCCCTGGGTACCCCGGTCGTCGTTCGTCCGGCCAACCTTCG
ACGGTTGGCCGAGCCGCTGATGGACTTACCCAAACCCACCCGCCCGATCGTGCGCA
CTAGGTCCTGTCGCTGCCCCCCAAACACCACCACGGGCCTGTTTGCGGAGGACAGCC
CCTTGGAGAGCACCGAGGTCGTGGACGCCGTGGCGTGCTTCCGACTGCTGCACCGA
GACCAACCCAGCCCCCCTCGCCTCTACCACTTGTGGGTGGTAGGCGCGGCGGATCTG
TGTGTGCCGTTTCTCGAATACGCCCAAAAAATCCGGCTCGGGGTAAGATTTATCGCC
ATCAAGACCCCAGACGCGTGGGTGGGAGAACCGTGGGCCGTGCCGACTCGGTTTTT
GCCCGAGTGGACCGTGGCGTGGACCCCGTTCCCCGCGGCCCCCAACCACCCCCTGG
AGACCCTGCTCAGCCGGTACGAATACCAGTACGGCGTGGTACTGCCCGGGACAAAC
GGACGGGAGCGCGATTGTATGCGCTGGCTGCGGTCCCTGATTGCTCTGCACAAACCC
CACCCAGCTACCCCAGGCCCCCTTACGACGTCCCATCCGGTGCGGCGTCCGTGTTGT
GCGTGTATGGGCATGCCCGAGATCCCAGACGAGCAACCCACATCACCGGGCCGTGG
TCCGCAAGAAACGGACCCTCTGATCGCCGTTCGCGTCGAACGGCCCAGACTTCCTCA
CATCTGCTATCCGGTTACCACCCTGTAG3′
SEQ ID NO: 3
DNA
HSV-1 US8
ATGGATCGCGGGGCGGTGGTGGGGTTTCTTCTCGGTGTTTGTGTTGTATCGTGCTTGG
CGGGAACGCCCAAAACGTCCTGGAGACGGGTGAGTGTCGGCGAGGACGTTTCGTTG
CTTCCAGCTCCGGGGCCTACGGGGCGCGGCCCGACCCAGAAACTACTATGGGCCGT
GGAACCCCTGGATGGGTGCGGCCCCTTACACCCGTCGTGGGTCTCGCTGATGCCCCC
CAAGCAGGTGCCCGAGACGGTCGTGGATGCGGCGTGCATGCGCGCTCCGGTCCCGC
TGGCGATGGCGTACGCCCCCCCGGCCCCATCTGCGACCGGGGGTCTACGGACGGAC
TTCGTGTGGCAGGAGCGCGCGGCCGTGGTTAACCGGAGTCTGGTTATTTACGGGGTC
CGAGAGACGGACAGCGGCCTGTATACCCTGTCTGTGGGCGACATAAAGGACCCGGC
TCGCCAAGTGGCCTCGGTGGTCCTGGTGGTGCAACCGGCCCCAGTTCCGACCCCACC
CCCGACCCCAGCCGATTACGACGAGGATGACAATGACGAGGGCGAGGGCGAGGAC
GAAAGTCTAGCCGGCACTCCCGCCAGCGGGACCCCCCGGCTCCCGCCTCCCCCCGCC
CCCCCGAGGTCTTGGCCCAGCGCCCCCGAAGTCTCACACGTGCGTGGGGTGACCGTG
CGTATGGAGACTCCGGAAGCTATCCTGTTTTCCCCCGGGGAGGCGTTTAGCACGAAC
GTCTCCATCCATGCCATCGCCCACGACGACCAGACCTACACCATGGACGTCGTCTGG
TTGAGGTTCGACGTGCCGACCTCGTGTGCCGAGATGCGAATATACGAATCGTGTCTG
TATCACCCGCAGCTCCCAGAGTGTCTGTCCCCGGCCGACGCTCCGTGCGCCGCGAGT
ACGTGGACGTCTCGCCTGGCCGTCCGCAGCTACGCGGGGTGTTCCAGAACAAACCC
CCCGCCGCGCTGTTCGGCCGAGGCTCACATGGAGCCCTTCCCGGGGCTGGCGTGGCA
GGCGGCCTCCGTCAATCTGGAGTTCCGGGACGCGTCCCCACAACACTCCGGCCTGTA
TCTGTGCGTGGTGTACGTCAACGACCATATTCACGCATGGGGCCACATTACCATCAG
CACCGCGGCGCAGTACCGGAACGCGGTGGTGGAACAGCCCCTCCCACAGCGCGGCG
CGGATTTGGCCGAGCCCACCCACCCGCACGTCGGGGCCCCTCCCCACGCGCCCCCAA
CCCACGGCGCCCTGCGGTTAGGGGCGGTGATGGGGGCCGCCCTGCTGCTGTCTGCGC
TGGGGTTGTCGGTGTGGGCGTGTATGACCTGTTGGCGCAGGCGTGCCTGGCGGGCGG
TTAAAAGCAGGGCCTCGGGTAAGGGGCCCACGTACATTCGCGTGGCCGACAGCGAG
CTGTACGCGGACTGGAGCTCGGACAGCGAGGGAGAACGCGACCAGGTCCCGTGGCT
GGCCCCCCCGGAGAGACCCGACTCTCCCTCCACCAATGGATCCGGCTTTGAGATCTT
ATCACCAACGGCTCCGTCTGTATACCCCCGTAGCGATGGGCATCAATCTCGCCGCCA
GCTCACAACCTTTGGATCCGGAAGGCCCGATCGCCGTTACTCCCAGGCCTCCGATTC
GTCCGTCTTCTGGTAA
SEQ ID NO: 4
DNA
Artificial Sequence HSV-1 US8_W116STOP
ATGGATCGCGGGGCGGTGGTGGGGTTTCTTCTCGGTGTTTGTGTTGTATCGTGCTTGG
CGGGAACGCCCAAAACGTCCTGGAGACGGGTGAGTGTCGGCGAGGACGTTTCGTTG
CTTCCAGCTCCGGGGCCTACGGGGCGCGGCCCGACCCAGAAACTACTATGGGCCGT
GGAACCCCTGGATGGGTGCGGCCCCTTACACCCGTCGTGGGTCTCGCTGATGCCCCC
CAAGCAGGTGCCCGAGACGGTCGTGGATGCGGCGTGCATGCGCGCTCCGGTCCCGC
TGGCGATGGCGTACGCCCCCCCGGCCCCATCTGCGACCGGGGGTCTACGGACGGAC
TTCGTGTGACAGGAGCGCGCGGCCGTGGTTAACCGGAGTCTGGTTATTTACGGGGTC
CGAGAGACGGACAGCGGCCTGTATACCCTGTCTGTGGGCGACATAAAGGACCCGGC
TCGCCAAGTGGCCTCGGTGGTCCTGGTGGTGCAACCGGCCCCAGTTCCGACCCCACC
CCCGACCCCAGCCGATTACGACGAGGATGACAATGACGAGGGCGAGGGCGAGGAC
GAAAGTCTAGCCGGCACTCCCGCCAGCGGGACCCCCCGGCTCCCGCCTCCCCCCGCC
CCCCCGAGGTCTTGGCCCAGCGCCCCCGAAGTCTCACACGTGCGTGGGGTGACCGTG
CGTATGGAGACTCCGGAAGCTATCCTGTTTTCCCCCGGGGAGGCGTTTAGCACGAAC
GTCTCCATCCATGCCATCGCCCACGACGACCAGACCTACACCATGGACGTCGTCTGG
TTGAGGTTCGACGTGCCGACCTCGTGTGCCGAGATGCGAATATACGAATCGTGTCTG
TATCACCCGCAGCTCCCAGAGTGTCTGTCCCCGGCCGACGCTCCGTGCGCCGCGAGT
ACGTGGACGTCTCGCCTGGCCGTCCGCAGCTACGCGGGGTGTTCCAGAACAAACCC
CCCGCCGCGCTGTTCGGCCGAGGCTCACATGGAGCCCTTCCCGGGGCTGGCGTGGCA
GGCGGCCTCCGTCAATCTGGAGTTCCGGGACGCGTCCCCACAACACTCCGGCCTGTA
TCTGTGCGTGGTGTACGTCAACGACCATATTCACGCATGGGGCCACATTACCATCAG
CACCGCGGCGCAGTACCGGAACGCGGTGGTGGAACAGCCCCTCCCACAGCGCGGCG
CGGATTTGGCCGAGCCCACCCACCCGCACGTCGGGGCCCCTCCCCACGCGCCCCCAA
CCCACGGCGCCCTGCGGTTAGGGGCGGTGATGGGGGCCGCCCTGCTGCTGTCTGCGC
TGGGGTTGTCGGTGTGGGCGTGTATGACCTGTTGGCGCAGGCGTGCCTGGCGGGCGG
TTAAAAGCAGGGCCTCGGGTAAGGGGCCCACGTACATTCGCGTGGCCGACAGCGAG
CTGTACGCGGACTGGAGCTCGGACAGCGAGGGAGAACGCGACCAGGTCCCGTGGCT
GGCCCCCCCGGAGAGACCCGACTCTCCCTCCACCAATGGATCCGGCTTTGAGATCTT
ATCACCAACGGCTCCGTCTGTATACCCCCGTAGCGATGGGCATCAATCTCGCCGCCA
GCTCACAACCTTTGGATCCGGAAGGCCCGATCGCCGTTACTCCCAGGCCTCCGATTC
GTCCGTCTTCTGGTAA

1. A modified HSV-1 vector, wherein the genome of the modified HSV-1 vector comprises an inactivating deletion in the coding sequence and/or the noncoding region of US8; and wherein the modified HSV-1 vector is able to grow in a suspension cell line.

2. The modified HSV-1 vector of claim 1, wherein the inactivating deletion comprises a single nucleotide substitution in the coding sequence of US8.

3. The modified HSV-1 vector of claim 1, wherein the parental HSV-1 vector is a human HSV-1 strain F.

4. The modified HSV-1 vector of claim 3, wherein the single nucleotide substitution is at nucleotide position 348 in the coding sequence of US8 and results in a W116STOP mutation, which nucleotide position is numbered relative to the position in the wild-type US8 gene (SEQ ID NO: 3).

5. The modified HSV-1 vector of claim 2, wherein the coding sequence of US8 after substitution comprises a nucleic acid sequence of SEQ ID NO: 4.

6. The modified HSV-1 vector of claim 1, wherein the modified HSV-1 vector is a replication competent HSV-1 vector, a defective helper-independent HSV-1 vector, a helper HSV-1 vector, or an HSV-1 amplicon vector.

7. The modified HSV-1 vector of claim 6, wherein the modified HSV-1 vector is an HSV-1 amplicon vector.

8. The HSV-1 amplicon vector of claim 7, wherein the modified HSV-1 amplicon vector comprises a mutant US8 protein, expressed by SEQ ID NO: 4, that comprises a W116STOP mutation relative to the wild-type US8 protein.

9. The HSV-1 amplicon vector of claim 1, wherein the HSV-1 amplicon vector comprises a non-functional US8 protein or lacks the US8 protein.

10. The modified HSV-1 vector according to claim 1, comprising a genome comprising an exogenous expression cassette.

11. The modified HSV-1 vector of claim 10, wherein the expression cassette comprises at least one nucleic acid sequence encoding a gene product.

12. A pharmaceutical composition comprising a modified HSV-1 vector according to claim 1 and a pharmaceutical excipient.

13. A kit comprising a modified HSV-1 vector according to claim 1 and instructions.

14. A method of producing an HSV-1 vector in a suspension cell line, the method comprising infecting a suspension cell line with the modified HSV-1 vector according to claim 1; and culturing the infected cells.

15. The method of claim 14, wherein the suspension cell line is selected from CHO, HeLa, H-9, Jurkat, C6/36, High Five, S2, Sf21, Sf9, PC-1, and HEK293.

16. The method of claim 15, wherein the suspension cell line is HEK293.

17. A method of producing an HSV-1 amplicon vector in a suspension cell line, wherein the method comprises infecting a suspension cell line with a helper virus packaging system that comprises: an HSV-1 amplicon vector or an HSV-1 amplicon plasmid and a helper HSV-1 vector; and wherein the helper HSV-1 vector comprises a US8 gene comprising a nucleic acid sequence of SEQ ID NO: 4 or an inactivating deletion of the US8 gene; and culturing the infected cells.

18. The method of claim 17, wherein the suspension cell line is selected from CHO, HeLa, H-9, Jurkat, C6/36, High Five, S2, Sf21, Sf9, PC-1, and HEK293.

19. The method of claim 18, wherein the suspension cell line is HEK293.


SEQUENCE LISTING (4 sequences)

SEQ ID NO: 1 - DNA, 876 bp, Human alphaherpesvirus 1
  source 1..876: mol_type = genomic DNA
atgggcgttgttgtcgtcaacgtaatgaccctccttgaccagaacaacgccctgccccggacttccgtcgacgcaagcccggccctgtggagcttcctgtttaggcagtgccgcattttggcatcagaacccctgggtaccccggtcgtcgttcgtccggccaaccttcgacggttggccgagccgctgatggacttacccaaacccacccgcccgatcgtgcgcactaggtcctgtcgctgccccccaaacaccaccacgggcctgtttgcggaggacagccccttggagagcaccgaggtcgtggacgccgtggcgtgcttccgactgctgcaccgagaccaacccagcccccctcgcctctaccacttgtgggtggtaggcgcggcggatctgtgtgtgccgtttctcgaatacgcccaaaaaatccggctcggggtaagatttatcgccatcaagaccccagacgcgtgggtgggagaaccgtgggccgtgccgactcggtttttgcccgagtggaccgtggcgtggaccccgttccccgcggcccccaaccaccccctggagaccctgctcagccggtacgaataccagtacggcgtggtactgcccgggacaaacggacgggagcgcgattgtatgcgctggctgcggtccctgattgctctgcacaaaccccacccagctaccccaggcccccttacgacgtcccatccggtgcggcgtccgtgttgtgcgtgtatgggcatgcccgagatcccagacgagcaacccacatcaccgggccgtggtccgcaagaaacggaccctctgatcgccgttcgcggcgaacggcccagacttcctcacatctgctatccggttaccaccctgtag

SEQ ID NO: 2 - DNA, 876 bp, synthetic construct
  source 1..876: mol_type = other DNA
atgggcgttgttgtcgtcaacgtaatgaccctccttgaccagaacaacgccctgccccggacttccgtcgacgcaagcccggccctgtggagcttcctgtttaggcagtgccgcattttggcatcagaacccctgggtaccccggtcgtcgttcgtccggccaaccttcgacggttggccgagccgctgatggacttacccaaacccacccgcccgatcgtgcgcactaggtcctgtcgctgccccccaaacaccaccacgggcctgtttgcggaggacagccccttggagagcaccgaggtcgtggacgccgtggcgtgcttccgactgctgcaccgagaccaacccagcccccctcgcctctaccacttgtgggtggtaggcgcggcggatctgtgtgtgccgtttctcgaatacgcccaaaaaatccggctcggggtaagatttatcgccatcaagaccccagacgcgtgggtgggagaaccgtgggccgtgccgactcggtttttgcccgagtggaccgtggcgtggaccccgttccccgcggcccccaaccaccccctggagaccctgctcagccggtacgaataccagtacggcgtggtactgcccgggacaaacggacgggagcgcgattgtatgcgctggctgcggtccctgattgctctgcacaaaccccacccagctaccccaggcccccttacgacgtcccatccggtgcggcgtccgtgttgtgcgtgtatgggcatgcccgagatcccagacgagcaacccacatcaccgggccgtggtccgcaagaaacggaccctctgatcgccgttcgcgtcgaacggcccagacttcctcacatctgctatccggttaccaccctgtag

SEQ ID NO: 3 - DNA, 1659 bp, Human alphaherpesvirus 1
  source 1..1659: mol_type = genomic DNA
atggatcgcggggcggtggtggggtttcttctcggtgtttgtgttgtatcgtgcttggcgggaacgcccaaaacgtcctggagacgggtgagtgtcggcgaggacgtttcgttgcttccagctccggggcctacggggcgcggcccgacccagaaactactatgggccgtggaacccctggatgggtgcggccccttacacccgtcgtgggtctcgctgatgccccccaagcaggtgcccgagacggtcgtggatgcggcgtgcatgcgcgctccggtcccgctggcgatggcgtacgcccccccggccccatctgcgaccgggggtctacggacggacttcgtgtggcaggagcgcgcggccgtggttaaccggagtctggttatttacggggtccgagagacggacagcggcctgtataccctgtctgtgggcgacataaaggacccggctcgccaagtggcctcggtggtcctggtggtgcaaccggccccagttccgaccccacccccgaccccagccgattacgacgaggatgacaatgacgagggcgagggcgaggacgaaagtctagccggcactcccgccagcgggaccccccggctcccgcctccccccgcccccccgaggtcttggcccagcgcccccgaagtctcacacgtgcgtggggtgaccgtgcgtatggagactccggaagctatcctgttttcccccggggaggcgtttagcacgaacgtctccatccatgccatcgcccacgacgaccagacctacaccatggacgtcgtctggttgaggttcgacgtgccgacctcgtgtgccgagatgcgaatatacgaatcgtgtctgtatcacccgcagctcccagagtgtctgtccccggccgacgctccgtgcgccgcgagtacgtggacgtctcgcctggccgtccgcagctacgcggggtgttccagaacaaaccccccgccgcgctgttcggccgaggctcacatggagcccttcccggggctggcgtggcaggcggcctccgtcaatctggagttccgggacgcgtccccacaacactccggcctgtatctgtgcgtggtgtacgtcaacgaccatattcacgcatggggccacattaccatcagcaccgcggcgcagtaccggaacgcggtggtggaacagcccctcccacagcgcggcgcggatttggccgagcccacccacccgcacgtcggggcccctccccacgcgcccccaacccacggcgccctgcggttaggggcggtgatgggggccgccctgctgctgtctgcgctggggttgtcggtgtgggcgtgtatgacctgttggcgcaggcgtgcctggcgggcggttaaaagcagggcctcgggtaaggggcccacgtacattcgcgtggccgacagcgagctgtacgcggactggagctcggacagcgagggagaacgcgaccaggtcccgtggctggcccccccggagagacccgactctccctccaccaatggatccggctttgagatcttatcaccaacggctccgtctgtatacccccgtagcgatgggcatcaatctcgccgccagctcacaacctttggatccggaaggcccgatcgccgttactcccaggcctccgattcgtccgtcttctggtaa

SEQ ID NO: 4 - DNA, 1659 bp, synthetic construct
  source 1..1659: mol_type = other DNA
atggatcgcggggcggtggtggggtttcttctcggtgtttgtgttgtatcgtgcttggcgggaacgcccaaaacgtcctggagacgggtgagtgtcggcgaggacgtttcgttgcttccagctccggggcctacggggcgcggcccgacccagaaactactatgggccgtggaacccctggatgggtgcggccccttacacccgtcgtgggtctcgctgatgccccccaagcaggtgcccgagacggtcgtggatgcggcgtgcatgcgcgctccggtcccgctggcgatggcgtacgcccccccggccccatctgcgaccgggggtctacggacggacttcgtgtgacaggagcgcgcggccgtggttaaccggagtctggttatttacggggtccgagagacggacagcggcctgtataccctgtctgtgggcgacataaaggacccggctcgccaagtggcctcggtggtcctggtggtgcaaccggccccagttccgaccccacccccgaccccagccgattacgacgaggatgacaatgacgagggcgagggcgaggacgaaagtctagccggcactcccgccagcgggaccccccggctcccgcctccccccgcccccccgaggtcttggcccagcgcccccgaagtctcacacgtgcgtggggtgaccgtgcgtatggagactccggaagctatcctgttttcccccggggaggcgtttagcacgaacgtctccatccatgccatcgcccacgacgaccagacctacaccatggacgtcgtctggttgaggttcgacgtgccgacctcgtgtgccgagatgcgaatatacgaatcgtgtctgtatcacccgcagctcccagagtgtctgtccccggccgacgctccgtgcgccgcgagtacgtggacgtctcgcctggccgtccgcagctacgcggggtgttccagaacaaaccccccgccgcgctgttcggccgaggctcacatggagcccttcccggggctggcgtggcaggcggcctccgtcaatctggagttccgggacgcgtccccacaacactccggcctgtatctgtgcgtggtgtacgtcaacgaccatattcacgcatggggccacattaccatcagcaccgcggcgcagtaccggaacgcggtggtggaacagcccctcccacagcgcggcgcggatttggccgagcccacccacccgcacgtcggggcccctccccacgcgcccccaacccacggcgccctgcggttaggggcggtgatgggggccgccctgctgctgtctgcgctggggttgtcggtgtgggcgtgtatgacctgttggcgcaggcgtgcctggcgggcggttaaaagcagggcctcgggtaaggggcccacgtacattcgcgtggccgacagcgagctgtacgcggactggagctcggacagcgagggagaacgcgaccaggtcccgtggctggcccccccggagagacccgactctccctccaccaatggatccggctttgagatcttatcaccaacggctccgtctgtatacccccgtagcgatgggcatcaatctcgccgccagctcacaacctttggatccggaaggcccgatcgccgttactcccaggcctccgattcgtccgtcttctggtaa